(19) United States
(12) Patent Application Publication
(10) Pub. No.: US 2021/0113891 A1
(43) Pub. Date: Apr. 22, 2021

(54) Adjustable Belay Optical Systems and Methods

(71) Applicant: Matthew Cupal, Logan, UT (US)

(72) Inventor: Matthew Cupal, Logan, UT (US)

(73) Assignee: PitchSix, LLC, Logan, UT (US)

(21) Appl. No.: 16/659,736

(22) Filed: Oct. 22, 2019

(51) Int. Cl. A63B 29/02; G02C 7/02; G02B 7/00; A61F 2/16

(52) U.S. Cl. CPC A63B 29/02; A61F 2/1648; G02B 7/002; G02C 7/02

(57) ABSTRACT

The present invention relates to a system for user adjustable optical field of view alteration. The system includes a support member, an optical system, and an adjustment system. The support member releasably secures the system to a user's face, including a frame over the user's nose and two temples extending back from the frame over the user's ears. The optical system is positioned on the frame substantially in front of the user's eyes. The optical system displays an altered field of view to the user's eyes that is different that the direct field of view seen by the user. The adjustment system adjusts the altered field of view by rotating a portion of the optical system with respect to the support member about an axis of rotation.

### RELATED APPLICATIONS

This application claims priority to U.S. provisional application Ser. No. 65/753,787 filed 10/31/18, the contents of which are incorporated by reference.

### FIELD OF THE INVENTION

The invention generally relates to adjustable belay optical systems and methods. In particular, the present invention relates to adjustable belay glasses.

### BACKGROUND OF THE INVENTION

Various multi-person activities include one stationary person controlling a rope that is attached to a second person who is ascending or descending a structure. The most common application is the activity of rock climbing, in which the stationary person is designated as the belayer and the ascending/descending person is designated as the climber. The belayer uses a belay device to feed and retract a rope that then extends above where it couples to the climber. The belay device is coupled to the belayer via a harness and the rope is likewise coupled to the climber via a harness. The act of belaying involves the belayer watching the climber and feeding or retracting the intercoupled rope through the belay device accordingly. The belayer must physically watch the climber to properly control the rope in a manner than allows the climber to efficiently ascend or descend the structure or rock face.

One of the major problems is that humans are not anatomically designed to look upward for long periods of time without straining or misaligning certain body parts. The prolonged act of looking upward by a belayer may result in a condition referred to as “belayer's neck”. Alternatively, a belayer may look away from the climber, compromising safety in an effort to prevent “belayer's neck”. One common solution to the “belayer's neck” problem is the use of prism-based glasses (hereinafter referred to as belay glasses). Belay glasses are analogous to conventional reading glasses in that they allow a user to look in a particular direction without moving their head. For example, belay glasses allow the belayer to see upward at a particular angle without necessarily requiring the belayer to tilt their head. A belayer may theoretically maintain a neutral neck position while continuously watching a climber, thus providing an optimal belay.

Although belay glasses have become widely adopted in the rock climbing industry, existing belay glasses suffer from a fundamental limitation that prevent optimal performance. It should be understood that belay glasses provide an additional field of view while obstructing part of the user's normal field of view (i.e. a belayer can still see peripherally around the belay glasses). Conventional belay glasses include a set of prisms mounted on a glasses frame such that the prisms are positioned in front of each user's eye. The prisms may be both 60° Bauerfeind prisms, meaning that a user looking horizontally will receive a field of view that is 30° below vertical. Unfortunately, the fixed field of view prevents optimal belaying in many circumstances. For example, modern rock climbing involves climbers ascending faces that vary significantly in steepness and the optimal belay position may vary in distance from the face. Therefore, a fixed angle which only displays a field of view that is 30° below vertical will inevitably force the belayer to either move their neck and potentially cause “belayer's neck”, stand in a suboptimal position in relation to climbing structure/face, and/or compromise the act of belaying in some other manner.

Therefore, there is a need in the industry for adjustable belay optical systems and methods.

### SUMMARY OF THE INVENTION

The present invention relates to adjustable optical systems and methods. One embodiment of the present invention relates to a system for user-adjustable optical field of view alteration. The system includes a support member, an optical system, and an adjustment system. The support member releasably secures the system to a user's face, including a frame over the user's nose and two temples extending back from the frame over the user's ears. The optical system is positioned on the frame substantially in front of the user's eyes. The optical system displays an altered field of view to the user's eyes that is different that the direct field of view seen by the user. The adjustment system adjusts the altered field of view by rotating a portion of the optical system with respect to the support member about an axis of rotation.

Embodiments of the present invention represent a significant advance in the field of user-adjusted optical field of view alteration. Conventional belay glasses and reading glasses alter a user's field of view for minimizing neck strain but fail to provide adjustability and are therefore inherently limited in their application. A set of conventional belay glasses or reading glasses will only optimally alter the field of view for a particular situation rather than the entire activity. Since the act of rock climbing and belaying involve a wide variety of climbing surfaces and belay regions, a fixed field of view alteration will not permit a belayer to optimally watch the climber during ascents of faces that exceed a particular angle or in situations that require the belayer to stand closer to/further from the climbing surface. Likewise, reading glasses are only designed for a particular anatomical orientation in which a user lies/sits and reads a book without looking down.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

The following description of the invention can be understood in light of the Figures, which illustrate specific aspects of the invention and are a part of the specification. Together with the following description, the Figures demonstrate and explain the principles of the invention. In the Figures, the physical dimensions may be exaggerated for clarity. The same reference numerals in different drawings represent the same element, and thus their descriptions will be omitted.

FIG. 1A illustrates an elevated perspective view of a system for user-adjustable optical field of view alteration in accordance with embodiments of the present invention;

FIG. 1B illustrates a bottom perspective view of a system for user-adjustable optical field of view alteration in accordance with embodiments of the present invention;

FIG. 2 illustrates an operational perspective view of a user wearing the system in FIGS. 1A and 1B;

FIG. 3 illustrates an exploded perspective view of the primary components of the system in FIGS. 1A and 1B;

FIG. 4 illustrates a partial transparent perspective detailed view of the adjustment system of the system in FIGS. 1A and 1B;

FIG. 5A illustrates a perspective detailed view of the adjustment system of the system in FIGS. 1A and 1B;

FIG. 5B illustrates a partial perspective detailed view of the adjustment system of the system in FIG. 5A; and

FIGS. 6A-D illustrate optical schematic operational views of one embodiment of the present invention adjusted in four different configurations.

### DETAILED DESCRIPTION OF THE INVENTION

The present invention relates to adjustable optical systems and methods. One embodiment of the present invention relates to a system for user adjustable optical field of view alteration. The system includes a support member, an optical system, and an adjustment system. The support member releasably secures the system to a user's face, including a frame over the user's nose and two temples extending back from the frame over the user's ears. The optical system is positioned on the frame substantially in front of the user's eyes. The optical system displays an altered field of view to the user's eyes that is different that the direct field of view seen by the user. The adjustment system adjusts the altered field of view by rotating a portion of the optical system with respect to the support member about an axis of rotation. Also, while embodiments are described in reference to belay glasses, it will be appreciated that the teachings of the present invention are applicable to other areas including but not limited to reading glasses and other activities which would benefit from an altered field of view.

The following terms are defined as follows:

### Definitions

Belay—the act of controlling a rope extending to another individual.

Belayer—the person performing the act of belaying.

Field of view—a restricted visual field within which a user may view. The term field of view is used throughout this application in reference to human optical viewing rather than any type of device or animal. Likewise, although human field of view is three dimensional, it will be appreciated that the field of view throughout this application in terms of a two-dimensional range. The system(s) of this application alter the vertical field of view orientation rather than a user's entire field of view (i.e. peripheral).

Altered field of view—an alternative field of view from that which the user's eyes see without neck movement. For example, if a user's neck is neutral, the user has a basic horizontal field of view in front of the user's eyes. An altered field of view would be a field of view oriented a specific angle up or down from the horizontal field of view of the user. Therefore, showing a user an altered field of view that is 30 degrees from vertical is altered 60 degrees from a user's horizontal field of view.

Anatomical reference vicinities—various anatomical terms are referenced on a user's face including but not limited to the face, nose, eyes, ears, etc. These terms are used for reference purposes only, and nothing herein constitutes any claim to human nature. In the claims, each anatomical term is referenced with the term “vicinity” to indicate that the system is disposed in relation to an anatomical region but does not in any way claim the human region.

Vertical and Horizontal—all orientations used throughout the application are in reference to a user's visual perception. Therefore, a vertical orientation is oriented vertically with respect to a user's eye (i.e. up-down) and a horizontal orientation is oriented horizontally with respect to a user's eye (i.e. side-side).

Axis of rotation—an axis about which one member rotates about another member.

For example, in the description below, various components are rotatable about other components about an axis of rotation.

Pivotable coupling—a coupling in which the axis of rotation is disposed substantially on one side of a rotating member and which is permitted to rotate towards and away from a fixed member. In contrast, a rotatable coupling is broader than a pivotable coupling because it does not require any proximity between the axis of rotation and the rotating member.

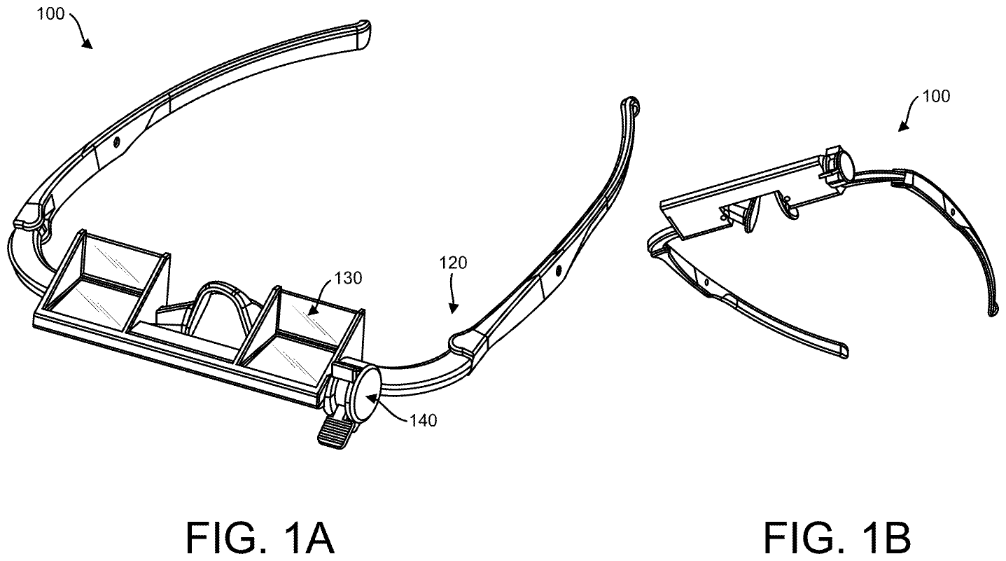

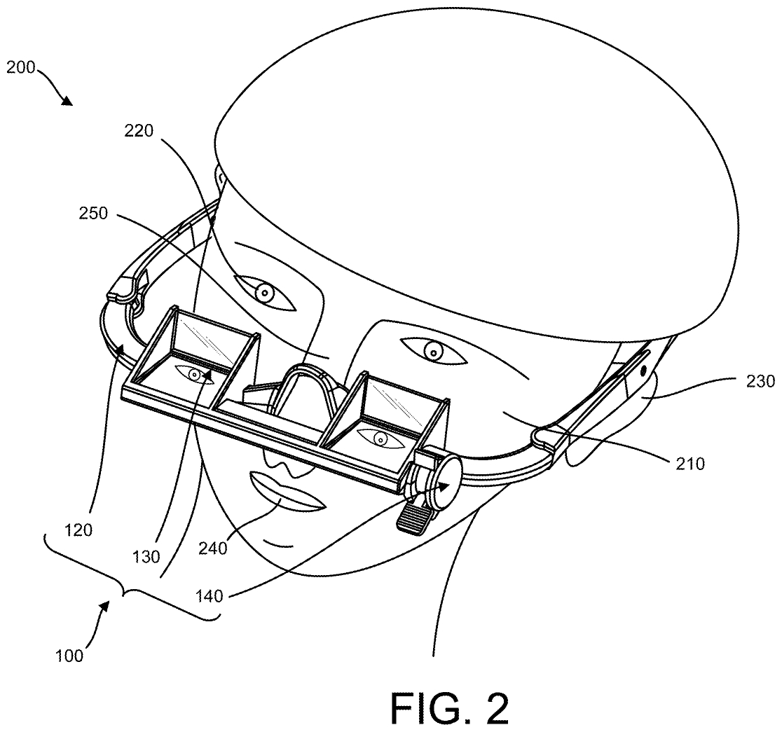

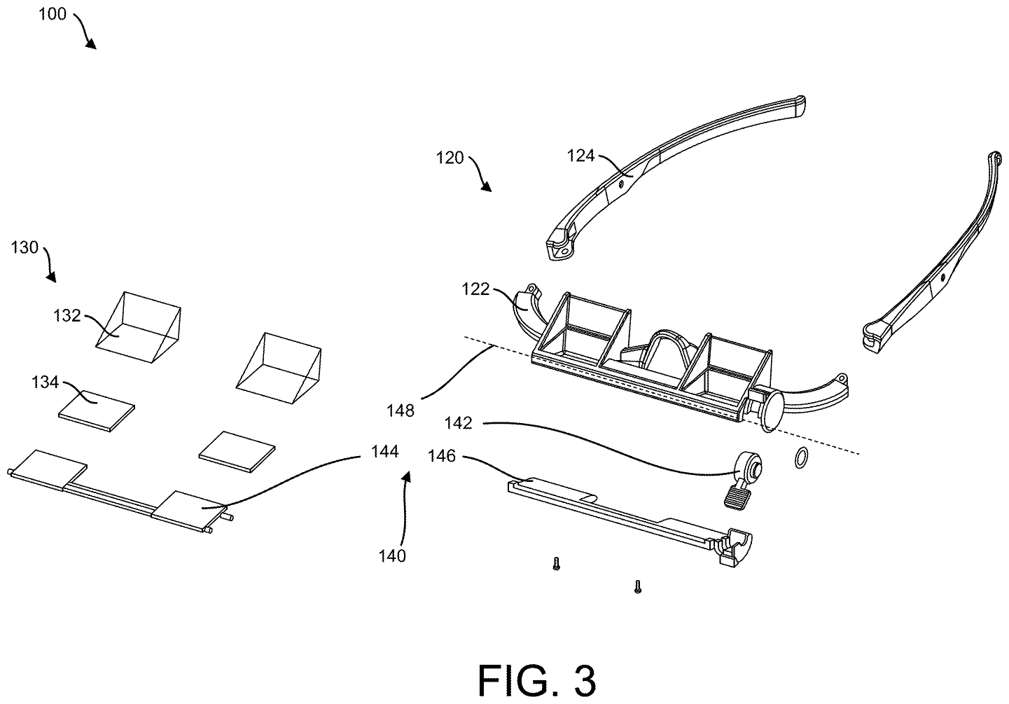

Reference is initially made to FIGS. 1-3, which illustrates perspective views of a system designated generally at 100. The illustrated system 100 is one embodiment of a system for user-adjustable optical field of view alteration. Various alternative embodiments will be described or referenced below. The following details are provided in relation to the illustrated system 100, but it should be appreciated that various alternative configurations have been both contemplated and described. The system 100 includes a support member 120, an optical system 130, and an adjustment system 140. The support member 120 is configured to support the system 100 in the vicinity of a user's face for purposes of optical enhancement during various activities, as shown in FIG. 2. For example, the system 100 is configured to provide a user with an adjustable altered field of view for the act of watching an elevated climber for long periods of time without incurring neck strain. The support member 120 may further include a frame 122 and two temples 124 (i.e. ear pieces). The frame 122 is configured to support a portion of the system in front of the user's face. As shown in FIG. 2, the frame 122 may include a nose support region. The two temples 124 are coupled to either side of the frame 122 and extend back from the user's face region over each of the user's ears respectively. The frame 122 is further configured to support a platform over the user's nose and substantially in front of the user's eyes. The two temples 124 further support this orientation by coupling to the frame 122 and supporting over each of the user's ears. This support configuration is analogous to many types of glasses and masks worn by people for various purposes. The frame 122 further provides a platform upon which the remainder of the system 100 may be disposed and oriented with respect to the user's face. In particular, the optical system 130 and adjustment system 140 are substantially supported by the support member 120 in the region in front of the user's eyes. The system 100 may further be configured to allow a user to maintain a standard field of view and a peripheral field of view during use of the system 100. For example, a user may be permitted to view around the system 100 to see the “naked” field of view which would otherwise be visible without the system. Likewise, a user may be permitted to maintain their “naked” peripheral view during use of the system 100 for safety and other purposes.

The system 100 further includes an optical system 130 which receives and displays an altered field of view for the user. The optical system 130 may includes two prisms 132 and two mirrors 134. It is well known that prisms receive and redirect light via reflection and refraction. In accordance with embodiments of the present invention, the optical system 130 and in particular the prisms 132 are configured to alter the field of view seen by the user. The prisms 132 and mirrors 134 operate in conjunction to provide an adjustable alteration of the field of view seen by the user. The mirrors 134 may replace or augment a reflective bottom surface of the prism and are configured to pivotably rotate away from the prims to adjustably alter the field of view displayed to the user. The rotation of the mirrors 134 with respect to the prisms 132 will be further described below in reference to the adjustment system 140. Although illustrated as two prisms 132 and two mirrors 134, it will be appreciated that alternative embodiments may utilize a single prism and/or a single mirrors as will be described in the alternative embodiments below. The optical system 130 is coupled to the frame 122 of the support member 120 so as to be disposed in front of each of the user's eyes. The two prisms 132 and two mirrors 134 are balanced and matched to minimize aberrations which may otherwise occur if discrepancies are present between the two mirrors 132 and prisms 134. Various alternative or optional balancing configurations are described below.

Various types of prisms and mirrors may be used in accordance with the present invention, but the illustrated embodiment includes a standard 30/60/90 prism and a set of mirrors. The region between the prisms 132 and mirrors 134 is filled with air in the illustrated embodiment, but it will be appreciated that alternative liquids or gases may be used to minimize aberrations. Likewise, the mirrors 134 and one or more of the surfaces of the prisms 132 may include optical coatings for purposes of minimizing reflections, glare, aberrations, etc.

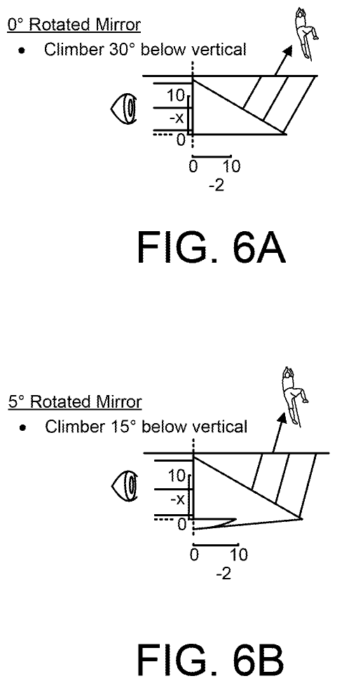

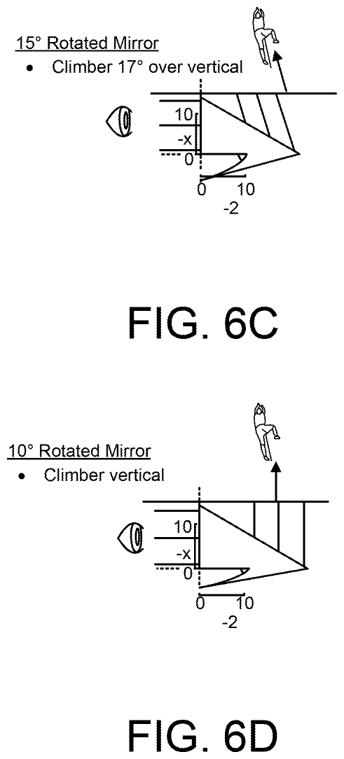

The system 100 includes an adjustment system 140 configured to adjust the altered field of view displayed to the user. The illustrated adjustment system 140 may include a lever 142, a mirror holder 144, a bottom cap 146, and an axis of rotation 148. The adjustment system 140 is configured to allow a user to adjust the pivotal angle between the mirrors 134 and the prisms 132 so as to adjust the field of view displayed to the user. In particular, the mirrors 134 are coupled to a mirror holder 144 which may be supported by a bottom cap 146. The mirror holder 144 is rotatably, and in particular, pivotably coupled to the frame 122 about the axis of rotation 148. The bottom cap 146 may be fixably coupled to the frame 122 so as to support the mirror holder 144. This configuration allows the mirrors 134 to be rotated/pivoted away from the prisms 132 about the axis of rotation 148. It will be appreciated that a particular angle is formed between the mirrors 134 and the prisms 132 since the axis of rotation 148. As discussed in reference to FIGS. 6A-D, this mirror-prism angle directly affects the altered field of view seen by the user. The lever 142 is coupled via a mechanism to both the mirror holder 144 and bottom cap 146 to facilitate user selection of the rotation of the mirrors 134 from the prisms 132. The mechanism between the lever 142 and the mirror holder 144 and bottom cap 146 is configured to securely allow the user to control the rotation by small increments. Various optional and alternative mechanisms incorporate a reliable step-down system and will be described below.

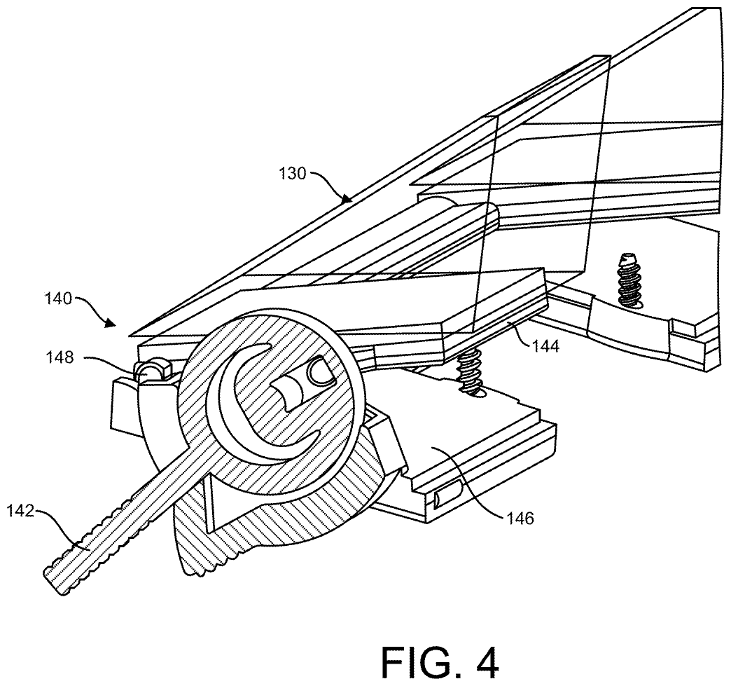

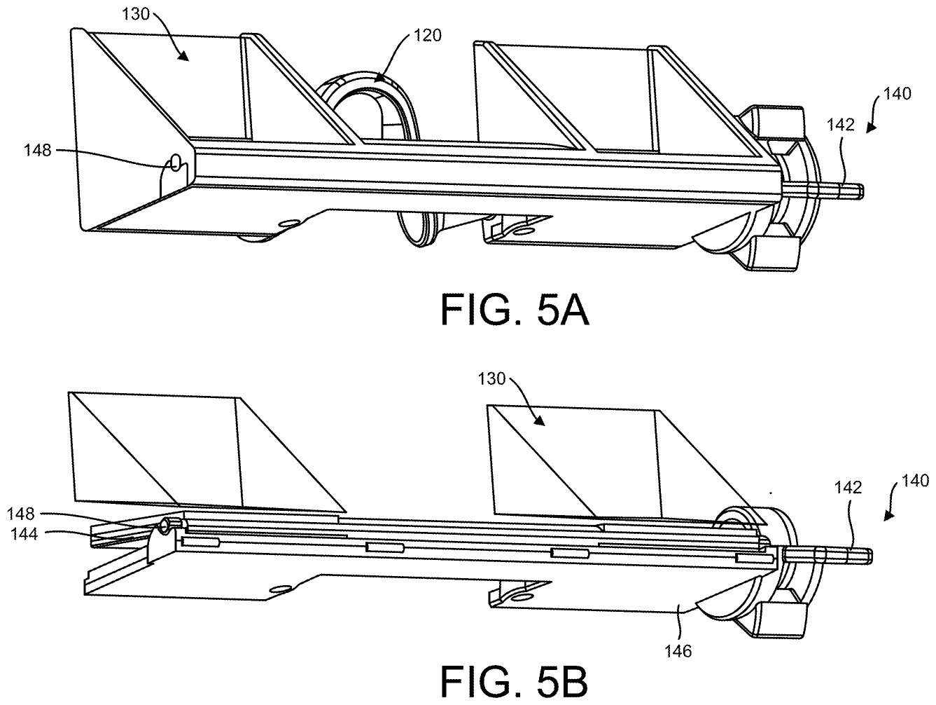

Reference is next made to FIGS. 4-5, which illustrate partial transparent perspective views of the adjustment system designated generally at 140. As described above, the lever 142 is coupled to a mechanism which controls the rotation of the mirror holder 144 with respect to the prisms 132 and bottom cap 146. Since the mirrors 134 are directly disposed on the mirror holder 144, the lever 142 fundamentally controls the rotation of the mirrors 134 with respect to the prisms 132 (mirror-prism angle). The specific illustrated mechanism includes a geometrical lever arm, pin, coupling spring, etc. for purposes of selectively controlling the angle between the mirrors 134 and the prisms 132. Another critical feature is the location of the axis of rotation 148 with respect to the remainder of the system 100. The axis of rotation 148 is uniquely positioned at the outer edge of the prisms 132 opposite to the user. This inherently allows the mirrors 134 to rotate toward the user. The lever 142 may also be oriented away from the user to allow for intuitive operation. For example, if the lever 142 was facing the user, it may not be visible during operation which would then require the user to remove the system 100 to make adjustments. The mechanism may also incorporate a particular step-down ratio such that rotating the lever 10 degrees may only rotate the mirror by one degree (10× step down). As discussed below, the mirror-prism angle has a dramatic affect on the field of view, and therefore if the mechanism does not step down the rotation, a user may be unable to properly select the intended field of view because of inherent dexterity limitations.

Reference is next made to FIGS. 6A-D, which illustrate optical schematic operational views of one embodiment of the present invention adjusted in four different mirror-prism angle configurations. As described above, the system 100 may allow a user to selectively alter the mirror-prism angle for purposes of altering the field of view. The illustrated optical schematics display how particular mirror-prism angles may alter the field of view of one embodiment of the system 100. It will be appreciated that various variables or factors may effect the resulting field of view; therefore, these figures are not intended to be accurate representations but merely examples of how a system 100 in accordance with embodiments of the present invention may be implemented in operation by a user. The illustrated examples reference a belayer engaged in the act of belaying a climber. The act of belaying a climber requires that the belayer watch the climber for long periods of time. As described above, one implementation of embodiments of the present invention is to allow the belayer to watch the climber without straining their neck. Therefore, the belayer may use the system 100 to alter their field of view so as to visually watch the climber without looking up which over time will cause neck strain. In particular, the belayer may adjust the system 100 to watch the climber at various angles. The illustrated schematics show how the rotation of the mirrors 134 with respect to the prisms 132 change the field of view and thereby allow the user to watch the climber at a wide variety of angles.

Various non-illustrated alternative or optional mechanisms may be incorporated into the adjustment system 140 to permit the user to selectively control the mirror-prism angle. For example, various direct, indirect, magnetic, geared, electrical, independent, and dependent mechanisms may be used independently or in combination with one or more of the other mechanisms. Likewise, various non-illustrated prism and mirror systems may be incorporated into the optical system 130 to minimize aberrations or distortion. For example, a sealed region between the mirror(s) and prism(s) may incorporate a liquid or gas that is matched to the type of glass used in the prism. Various non-illustrated mirror alignment systems or mechanisms may also be incorporated into the optical system 130 to minimize aberrations or distortion. The user may be allowed to manually adjust each mirror-prism angle independently. Various types of couplings could extend between the mirrors to permit rotation and maintain consistent alignment. The surfaces of the prisms 132 may also include various coatings or curvatures to minimize aberrations or distortion. Various non-illustrated support member configurations may also be incorporated in accordance with embodiments of the present invention for particular applications. Various peripheral blocking structures may be incorporated if the user's peripheral vision is intended to be blocked. The temples or ear pieces may be configured to fold or bend toward the frame to facilitate compact storage. Finally, a set of sunglass lenses, magnifying lenses, or corrective lenses may optionally couple to the prisms to prevent glare.

It should be noted that various alternative system designs may be practiced in accordance with the present invention, including one or more portions or concepts of the embodiment illustrated in FIG. 1 or described above. Various other embodiments have been contemplated, including combinations in whole or in part of the embodiments described above.

What is claimed is:

1. A system for user adjustable optical field of view alteration comprising:
a support member configured to releasably secure the system to a user's face vicinity including a frame and two temples;
an optical system disposed on the frame in correspondence with a user's eye vicinity, wherein the optical system is configured to display an altered field of view; and
an adjustment system coupled to the support member and the optical system, wherein the adjustment system is configured to adjust the altered field of view via rotating a portion of the optical system with respect to the support member about an axis of rotation, wherein a range of rotation of the portion of the optical system with respect to the support member corresponds to an adjustable altered field of view range.

2. The system of claim 1, wherein the altered field of view range is between 30 degrees below vertical to 8 degrees above vertical.

3. The system of claim 1, wherein the optical system includes two 30/60/90 prisms.

4. The system of claim 1, wherein the optical system includes two prisms and two mirrors disposed on the frame, and wherein the frame is configured such that the two prisms are disposed substantially in a user's eye vicinity, and wherein the two mirrors are disposed below each of the two prisms, respectively.

5. The system of claim 4, wherein the two prisms each include a short side, medium side, and long side, and wherein the short side of each prism is disposed vertically in front of the user's eye vicinity, and wherein the long side of each prism is disposed horizontally on the frame, and wherein the medium side of each prism is disposed on the frame opposite of the temples.

6. The system of claim 1, wherein the adjustment system includes a lever and a mirror holder rotatably coupled to the frame and a bottom cap.

7. The system of claim 6, wherein mirror holder and bottom cap extend lengthwise substantially parallel to a lengthwise orientation of the frame between the two temples.

8. The system of claim 7, wherein the mirror holder is pivotably coupled to the frame about the axis of rotation, and wherein the mirror holder and bottom cap are disposed below the optical system.

9. The system of claim 8, wherein the two mirrors are coupled to the mirror holder, and wherein the two mirrors are vertically aligned below the two prisms.

10. The system of claim 6, wherein the lever is rotatably coupled to the frame and fixably coupled to the mirror holder such that rotating the lever with respect to the frame causes the mirror holder to correspondingly rotate with respect to the frame.

11. The system of claim 1, wherein the axis of rotation is disposed opposite the two temples.

12. The system of claim 1, wherein optical system includes the two prisms that each further include a short side, medium side, and long side, and wherein the short side of each prism is disposed vertically in the user's eye vicinity, and wherein the axis of rotation is vertically aligned below a corner of the prism opposite the short side.

13. The system of claim 1, wherein the optical system includes two prisms and one mirror disposed on the frame, and wherein the frame is configured such that the two prisms are disposed substantially in the user's eye vicinity, and wherein the one mirror is disposed below both of the two prisms.

14. The system of claim 1, wherein the optical system includes two optical detectors and two optical displays, and wherein the frame is configured such that the two optical displays are disposed substantially in the user's eye vicinity.

15. The system of claim 14, wherein the adjustment system is rotatably coupled to the frame and fixably coupled to the two optical detectors such that rotating a lever of the adjustment system with respect to the frame causes the two optical detectors to correspondingly rotate with respect to the frame.

16. The system of claim 1, wherein the axis of rotation is parallel to a lengthwise orientation of the frame and extends between the two temples.

17. The system of claim 1, wherein the two temples are configured to extend from the frame across the user's face vicinity and over each of a user's ear vicinities.

18. The system of claim 1, wherein the frame is substantially orthogonal to the two temples.

19. A system for user adjustable optical field of view alteration comprising:
a support member configured to releasably secure the system to a user's face vicinity including a frame and two temples;
an optical system disposed on the frame in correspondence with the user's eye vicinity, wherein the optical system is configured to display an altered field of view; and
an adjustment system coupled to the support member and the optical system, wherein the adjustment system is configured to adjust the altered field of view via rotating a portion of the optical system with respect to the support member about an axis of rotation, wherein a range of rotation of the portion of the optical system with respect to the support member corresponds to an adjustable altered field of view range, and wherein the axis of rotation is opposite the two temples.

20. A system for user adjustable optical field of view alteration comprising:
a support member configured to releasably secure the system to a user's face vicinity including a frame and two temples, wherein the frame is disposed in a user's eye vicinity and the two temples extend from the frame over a user's ear vicinity;
an optical system disposed on the frame in correspondence with the user's eye vicinity, wherein the optical system is configured to display an altered field of view; and an adjustment system coupled to the support member and the optical system, wherein the adjustment system is configured to adjust the altered field of view via rotating a portion of the optical system with respect to the support member about an axis of rotation, wherein a range of rotation of the portion of the optical system with respect to the support member corresponds to an adjustable altered field of view range between 30 degrees below vertical to 8 degrees above vertical.
